(19) United States
(12) Patent Application Publication
(10) Pub. No.: US 2003/0149062 A1
(43) Pub. Date: Aug. 7, 2003

(54) Use of tyrosine kinase inhibitors for the treatment of inflammatory processes

(75) Inventors: Birgit Jung, Laupheim (DE); Hubert Pueschner, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/353,616

(22) Filed: Jan. 29, 2003

(30) Foreign Application Priority Data
Feb. 5, 2002 (DE) DE 102 04 462.7

(51) Int. Cl. A61K 31/517; A61K 31/4709; A61K 31/55

(52) U.S. Cl. 514/266.22; 514/314; 514/313; 514/266.24; 514/266.4; 514/217.06

(57) ABSTRACT

A method of treating inflammatory diseases of the airways or intestines which comprises administering substances selected from the group consisting of:

(a) quinazolines of general formula

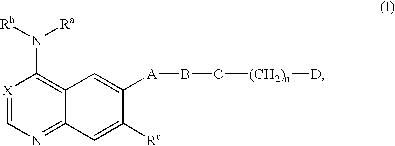

wherein A, B, C, D, X, R^{a}, R^{b}, R^{c} and n are as defined herein,

(b) the compounds

(1) 4-[(3-chloro-4-fluorophenyl)amino]-6-[(4-dimethylamino-cyclohexyl)amino]-pyrimido[5,4-d]pyrimidine,

(2) 4-[(R)-(1-phenylethyl)amino]-6-(4-hydroxyphenyl)-7H-pyrrolo[2,3-d]pyrimidine, and

(3) 4-{[3-chloro-4-(3-fluoro-4-benzyloxy)-phenyl]amino}-6-(5-{[(2-methanesulphonyl-ethyl)amino]methyl}-furan-2-yl)quinazoline or

(d) the antibodies Cetuximab C225, Trastuzumab, ABX-EGF and Mab ICR-62, and

(f) EGFR-antisense.

### DESCRIPTION OF THE INVENTION

The present invention relates to the use of quinazolines of general formula

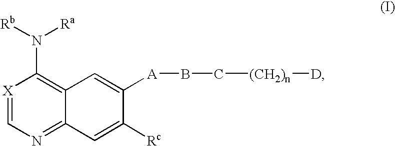

wherein A, B, C, D, X, R^{a}, R^{b}, R^{c} and n are defined below, or

the compounds

(1) 4-[(3-chloro-4-fluorophenyl)amino]-6-[(4-dimethylamino-cyclohexyl)amino]-pyrimido [5,4-d]pyrimidine,

(2) 4-[(R)-(1-phenylethyl)amino]-6-(4-hydroxyphenyl)-7H-pyrrolo[2,3-d]pyrimidine,

(3) 4-{[3-chloro-4-(3-fluoro-4-benzyloxy)-phenyl]amino}-6-(5-{[(2-methanesulphonyl-ethyl)amino]methyl}-furan-2-yl)quinazoline or

the antibody Cetuximab C225, Trastuzumab, ABX-EGF, Mab ICR-62 or EGFR-antisense,

the tautomers, stereoisomers and salts thereof, particularly the physiologically acceptable salts thereof with inorganic or organic acids or bases, for preparing a pharmaceutical composition for the prevention and treatment of

diseases of the airways or lungs which are accompanied by increased or altered production of mucus, such as e.g. inflammatory diseases of the airways such as acute bronchitis, chronic bronchitis, chronic obstructive bronchitis (COPD), asthma, bronchiectasis, allergic or non-allergic rhinitis or sinusitis, cystic fibrosis, α1-antitrypsin deficiency, coughs, lung emphysema, pulmonary fibrosis or hyperreactive airways.

Moreover, the compounds are also suitable for the treatment of inflammatory diseases of the gastro-intestinal tract or bile duct or gall bladder which are accompanied by impaired tyrosine kinase function, such as may be found for example in acute or chronic inflammatory changes such as cholecystitis, Crohn's disease, ulcerative colitis, ulcers or polyposis in the gastro-intestinal tract or such as occur in diseases of the gastro-intestinal tract which are associated with increased secretion, such as Ménétrier's disease, secreting adenomas or protein loss syndrome,

and also for the treatment of inflammatory diseases of the joints, such as rheumatoid arthritis, inflammatory diseases of the skin and the eyes, inflammatory pseudopolyps, in colitis cystica profunda or in pneumatosis cystoides intestinalis.

Preferred fields of application are inflammatory diseases of the respiratory tract or bowel, such as chronic bronchitis (COPD), chronic sinusitis, asthma, Crohn's disease, ulcerative colitis or polyposis of the intestines.

Particularly preferred fields of application are inflammatory diseases of the airways or lungs such as chronic bronchitis (COPD) or asthma.

In the above general formula (I)

X denotes a nitrogen atom or a carbon atom substituted by a cyano group,

R^{a} denotes a hydrogen atom or a C₁₋₄-alkyl group,

R^{b} denotes a phenyl, benzyl or 1-phenylethyl group, wherein the phenyl nucleus may be substituted in each case by the groups R¹ and R², while

R¹ and R², which may be identical or different, in each case denote a hydrogen, fluorine, chlorine, bromine or iodine atom,

a C₁₋₄-alkyl, hydroxy, C₁₋₄-alkoxy, C₃₋₆-cycloalkyl, C₄₋₆-cycloalkoxy, C₂₋₅-alkenyl or C₂₋₅-alkynyl group,

an aryl, aryloxy, arylmethyl or arylmethoxy group,

a C₃₋₅-alkenyloxy or C₃₋₅-alkynyloxy group, while the multiple bond is isolated from the oxygen atom,

a C₁₋₄-alkylsulphenyl, C₁₋₄-alkylsulphinyl, C₁₋₄-alkylsulphonyl, C₁₋₄-alkylsulphonyl-oxy, trifluoromethylsulphenyl, trifluoromethylsulphinyl or trifluoromethylsulphonyl group,

a methyl or methoxy group substituted by 1 to 3 fluorine atoms,

an ethyl or ethoxy group substituted by 1 to 5 fluorine atoms,

a cyano or nitro group or an amino group optionally substituted by one or two C₁₋₄-alkyl groups, while the substituents may be identical or different,

A denotes an oxygen atom or an imino group optionally substituted by a C₁₋₄-alkyl group,

B denotes a bond, a carbonyl or sulphonyl group,

C denotes a methylene, ethylene or ethenylene group,

n denotes one of the numbers 0 or 1,

D denotes an amino, C₁₋₄-alkylamino, C₃₋₅-cycloalkylamino or di-(C₁₋₄-alkyl)-amino or di-(C₃₋₅-cycloalkyl)-amino group wherein the alkyl and cycloalkyl moieties may be identical or different,

a C₂₋₄-alkylamino group wherein the alkyl moiety is substituted in the β, γ or δ position to the nitrogen atom of the amino group by the group R³, while

R³ denotes a hydroxy, C₁₋₄-alkoxy, C₁₋₃-alkoxycarbonyl, amino, C₁₋₄-alkylamino or di-(C₁₋₄-alkyl)-amino group,

a 4- to 7-membered alkyleneimino group optionally substituted by one or two methyl groups or

a 6- to 7-membered alkyleneimino group optionally substituted by one or two methyl groups wherein in each case a methylene group in the 4 position is replaced by an oxygen or sulphur atom, by a sulphinyl, sulphonyl, imino or N-(C₁₋₄-alkyl)-imino group,

an N—(C₁₋₄-alkyl)—N—(C₂₋₄-alkyl)-amino group wherein the alkyl moieties in the β, γ or δ position to the nitrogen atom of the amino group may optionally be substituted by the group R³, where R³ is as hereinbefore defined,

a di-(C₂₋₄-alkyl)-amino group wherein the two C₂₋₄-alkyl moieties in each case are substituted in the β, γ or δ position to the nitrogen atom of the amino group by the group R³, while the substituents may be identical or different and R³ is as hereinbefore defined,

a C₃₋₇-cycloalkylamino or C₃₋₇-cycloalkyl-C₁₋₃-alkylamino group, wherein in each case the nitrogen atom may be substituted by a further C₁₋₄-alkyl group,

an amino or C₁₋₄-alkylamino group, wherein in each case the nitrogen atom is substituted by a tetrahydrofuran-3-yl, tetrahydropyran-3-yl, tetrahydropyran-4-yl, tetrahydrofuranylmethyl, 1-(tetrahydrofuran-3-yl)-piperidin-4-yl, 1-(tetrahydropyran-3-yl)-piperidin-4-yl, 1-(tetrahydropyran-4-yl)-piperidin-4-yl, 3-pyrrolidinyl, 3-piperidinyl, 4-piperidinyl, 3-hexahydro-azepinyl or 4-hexahydro-azepinyl group optionally substituted by 1 to 3 C₁₋₄-alkyl groups,

a 4- to 7-membered alkyleneimino group optionally substituted by 1 to 4 C₁₋₂-alkyl groups which may be substituted by the group R³ either at a cyclic carbon atom or at one of the alkyl groups, while R³ is as hereinbefore defined,

a piperidino group substituted by a tetrahydrofuranyl, tetrahydropyranyl or tetrahydrofuranylmethyl group,

a 6- to 7-membered alkyleneimino group optionally substituted by 1 or 2 C₁₋₂-alkyl groups wherein in each case a methylene group in the 4 position is replaced by an oxygen or sulphur atom, by an imino group substituted by the group R⁴, by a sulphinyl or sulphonyl group, while

R⁴ denotes a hydrogen atom, a C₁₋₄-alkyl, 2-methoxy-ethyl, 3-methoxy-propyl, C₃₋₇-cycloalkyl, C₃₋₇-cycloalkyl-C₁₋₄-alkyl, tetrahydrofuran-3-yl, tetrahydropyran-3-yl, tetrahydropyran-4-yl, tetrahydrofuranylmethyl, formyl, C₁₋₄-alkylcarbonyl, C₁₋₄-alkylsulphonyl, aminocarbonyl, C₁₋₄-alkylaminocarbonyl or di-(C₁₋₄-alkyl)-aminocarbonyl group,

a morpholino or 2-oxo-morpholin-4-yl group which may be substituted by a methyl, ethyl or C₁₋₃-alkoxymethyl group,

an imidazolyl group optionally substituted by 1 to 3 methyl groups,

a C₅₋₇-cycloalkyl group wherein a methylene group is replaced by an oxygen or sulphur atom, by an imino group substituted by the group R⁴, by a sulphinyl or sulphonyl group, while R⁴ is as hereinbefore defined,

a hydroxy or C₁₋₄-alkoxy group, or also

a hydrogen atom, if n is the number 0, and

R^{c} denotes a hydrogen atom, a C₁₋₄-alkoxy-C₁₋₄-alkoxy, C₁₋₄-alkoxy, C₄₋₇-cycloalkoxy or C₃₋₇-cycloalkyl-C₁₋₆-alkoxy group, wherein the cycloalkyl moiety may be substituted in each case by a C₁₋₃-alkyl, hydroxy, C₁₋₄-alkoxy, amino, C₁₋₄-alkylamino, di-(C₁₋₄-alkyl)-amino, pyrrolidino, piperidino, morpholino, piperazino, N-(C₁₋₂-alkyl)-piperazino, hydroxy-C₁₋₂-alkyl, C₄-alkoxy-C₁₋₂-alkyl, amino-C₁₋₂-alkyl, C₁₋₄-alkylamino-C₁₋₂-alkyl, di-(C₁₋₄-alkyl)-amino-C₁₋₂-alkyl, pyrrolidino-C₁₋₂-alkyl, piperidino-C₁₋₂-alkyl, morpholino-C₁₋₂-alkyl, piperazino-C₁₋₂-alkyl or N-(C₁₋₂-alkyl)-piperazino-C₁₋₂-alkyl group, while the abovementioned monosubstituted cycloalkyl moieties may additionally be substituted by a C₁₋₃-alkyl group, or

a 3-pyrrolidinyloxy, 2-pyrrolidinyl-C₁₋₄-alkyloxy, 3-pyrrolidinyl-C₁₋₄-alkyloxy, 3-piperidinyloxy, 4-piperidinyloxy, 2-piperidinyl-C₁₋₄-alkyloxy, 3-piperidinyl-C₁₋₄-alkyloxy, 4-piperidinyl-C₁₋₄-alkyloxy, 3-hexahydro-azepinyloxy, 4-hexahydro-azepinyloxy, 2-hexahydro-azepinyl-C₁₋₄-alkyloxy, 3-hexahydro-azepinyl-C₁₋₄-alkyloxy or 4-hexahydro-azepinyl-C₁₋₄-alkyloxy group, wherein in each case the cyclic nitrogen atom is substituted by the group R⁴, where R⁴ is as hereinbefore defined,

a piperazino or homopiperazino group substituted in the 4 position by an R⁶-C₁₋₄-alkyl, R⁶—CO, R⁶—C₁₋₄-alkylene-CO, (R⁵NR⁷)-C₁₋₄-alkylene-CO, R⁷O—C₁₋₄-alkylene-CO R⁷S—C₁₋₄-alkylene-CO, R⁷SO—C₁₋₄-alkylene-CO or R⁷SO₂-C₁₋₄-alkylene-CO group, wherein

R⁵ denotes a hydrogen atom or a C₁₋₄-alkyl group,

R⁶ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups and

R⁷ denotes a 2-oxo-tetrahydrofuran-3-yl, 2-oxo-tetrahydrofuran-4-yl, 2-oxo-tetrahydropyran-3-yl, 2-oxo-tetrahydropyran-4-yl or 2-oxo-tetrahydropyran-5-yl group optionally substituted by one or two C₁₋₂-alkyl groups,

a morpholino-C₁₋₄-alkoxy or 2-oxo-morpholin-4-yl-C₁₋₆-alkoxy group which may be substituted by 1 or 2 methyl or ethyl groups, or

a tetrahydrofuran-3-yloxy, tetrahydropyran-3-yloxy, tetrahydropyran-4-yloxy, tetrahydrofuranylmethoxy or tetrahydropyranylmethoxy group.

By the aryl moieties mentioned in the definition of the abovementioned groups is meant a phenyl group, which may in each case be monosubstituted by R⁸, mono-, di- or trisubstituted by R⁹ or monosubstituted by R⁸ and additionally mono- or disubstituted by R⁹, while the substituents may be identical or different, while

R⁸ denotes a cyano, carboxy, C₁₋₄-alkoxycarbonyl, aminocarbonyl, C₁₋₄-alkyl-aminocarbonyl, di-(C₁₋₄-alkyl)-aminocarbonyl, C₁₋₄-alkylsulphenyl, C₁₋₄-alkylsulphinyl, C₁₋₄-alkylsulphonyl, hydroxy, C₁₋₄-alkylsulphonyloxy, trifluoromethyloxy, nitro, amino, C₁₋₄-alkylamino, di-(C₁₋₄-alkyl)-amino, C₁₋₄-alkylcarbonylamino, N-(C₁₋₄-alkyl)-C₁₋₄-alkylcarbonylamino, C₁₋₄-alkylsulphonylamino, N—(C₁₋₄-alkyl)-C₁₋₄-alkylsulphonylamino, aminosulphonyl, C₁₋₄-alkylaminosulphonyl or di-(C₁₋₄-alkyl)-aminosulphonyl group or a carbonyl group which is substituted by a 5- to 7-membered alkyleneimino group, while in the abovementioned 6- to 7-membered alkyleneimino groups in each case a methylene group in the 4 position may be replaced by an oxygen or sulphur atom, by a sulphinyl, sulphonyl, imino or N-(C₁₋₄-alkyl)-imino group, and

R⁹ denotes a fluorine, chlorine, bromine or iodine atom, a C₁₋₄-alkyl, trifluoromethyl or C₁₋₄-alkoxy group.

A preferred object of the invention is the use of the compounds of general formula (I) wherein

X denotes a nitrogen atom or a carbon atom substituted by a cyano group,

R^{a} denotes a hydrogen atom or a C₁₋₄-alkyl group,

R^{b} denotes a phenyl, benzyl or 1-phenylethyl group, wherein the phenyl nucleus may be substituted in each case by the groups R¹ and R², while

R¹ and R², which may be identical or different, in each case denote a hydrogen, fluorine, chlorine or bromine atom,

a C₁₋₄-alkyl, hydroxy, C₁₋₄-alkoxy, C₃₋₆-cycloalkyl, C₄₋₆-cycloalkoxy, C₂₋₅-alkenyl or C₂₋₅-alkynyl group,

a methyl, trifluoromethyl or methoxy group,

A denotes an oxygen atom or an imino group optionally substituted by a C₁₋₄-alkyl group,

B denotes a bond or a carbonyl group,

C denotes a methylene, ethylene or ethenylene group,

n denotes one of the numbers 0 or 1,

D denotes a di-(C₁₋₄-alkyl)-amino group wherein the alkyl moieties may be identical or different,

an N—(C₁₋₄-alkyl)—N—(C₂₋₄-alkyl)-amino group wherein the alkyl moieties in the β, γ or δ position to the nitrogen atom of the amino group may optionally be substituted by the group R³, while

R³ denotes a hydroxy, C₁₋₃-alkoxy, C₁₋₃-alkoxycarbonyl, amino, C₁₋₄-alkylamino or di-(C₁₋₄-alkyl)-amino group,

a pyrrolidino, piperidino or morpholino group,

a di-(C₂₋₄-alkyl)-amino group wherein the two C₂₋₄-alkyl moieties are substituted in each case in the β, γ or δ position to the nitrogen atom of the amino group by the group R³, while the substituents may be identical or different and R³ is as hereinbefore defined,

a C₃₋₅-cycloalkylamino or C₃₋₅-cycloalkyl-C₁₋₃-alkylamino group, wherein in each case the nitrogen atom is substituted by a further C₁₋₄-alkyl group,

a C₁₋₄-alkylamino group wherein the nitrogen atom is substituted by a tetrahydrofuran-3-yl, tetrahydropyran-3-yl, tetrahydropyran-4-yl, tetrahydrofuranylmethyl, 1-(tetrahydrofuran-3-yl)-piperidin-4-yl, 1-(tetrahydropyran-3-yl)-piperidin-4-yl or 1-(tetrahydropyran-4-yl)-piperidin -4-yl group,

a 5- to 7-membered alkyleneimino group optionally substituted by 1 to 2 methyl groups which may be substituted by the group R³ either at a cyclic carbon atom or at one of the methyl groups, while R³ is as hereinbefore defined,

a piperidino group substituted by a tetrahydrofuranyl, tetrahydropyranyl or tetrahydrofuranylmethyl group,

an piperidino group optionally substituted by 1 or 2 methyl groups wherein the methylene group in the 4 position is replaced by an oxygen or sulphur atom, by an imino group substituted by the group R⁴, by a sulphinyl or sulphonyl group, while

R⁴ denotes a hydrogen atom, a C₁₋₃-alkyl, 2-methoxy-ethyl, 3-methoxy-propyl, C₃₋₆-cycloalkyl, C₃₋₆-cycloalkyl-C₁₋₃-alkyl, tetrahydrofuran-3-yl, tetrahydropyran-3-yl, tetrahydropyran-4-yl, tetrahydrofuranylmethyl, C₁₋₃-alkylcarbonyl, C₁₋₃-alkylsulphonyl, aminocarbonyl, C₁₋₃-alkylaminocarbonyl or di-(C₁₋₃-alkyl)-aminocarbonyl group,

a morpholino or 2-oxo-morpholin-4-yl group which may be substituted by a methyl, ethyl or C₁₋₃-alkoxymethyl group,

a C₅₋₆-cycloalkyl group wherein a methylene group is replaced by an oxygen or sulphur atom, by an imino group substituted by the group R⁴, by a sulphinyl or sulphonyl group, while R⁴ is as hereinbefore defined,

a hydroxy or C₁₋₄-alkoxy group, or also

a hydrogen atom, if n is the number 0, and

R^{c} denotes a hydrogen atom, a C₁₋₄-alkoxy-C₁₋₄-alkoxy, C₁₋₄-alkoxy, C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group,

a 3-pyrrolidinyloxy, 2-pyrrolidinyl-C₁₋₃-alkyloxy, 3-pyrrolidinyl-C₁₋₃-alkyloxy, 3-piperidinyloxy, 4-piperidinyloxy, 2-piperidinyl-C₁₋₃-alkyloxy, 3-piperidinyl-C₁₋₃-alkyloxy or 4-piperidinyl-C₁₋₃-alkyloxy group, wherein in each case the cyclic nitrogen atom is substituted by the group R⁴, where R⁴ is as hereinbefore defined,

a piperazino or homopiperazino group substituted in the 4 position by an R⁶-C₁₋₄-alkyl, R⁶—CO or R⁶—C₁₋₄-alkylene-CO group, wherein

R⁶ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups,

a morpholino-C₁₋₄-alkoxy or 2-oxo-morpholin-4-yl-C₁₋₆-alkoxy group which may be substituted by 1 or 2 methyl or ethyl groups, or

a tetrahydrofuran-3-yloxy, tetrahydropyran-3-yloxy, tetrahydropyran-4-yloxy, tetrahydrofuranylmethoxy or tetrahydropyranylmethoxy group, or

the compounds

(1) 4-[(3-chloro-4-fluorophenyl)amino]-6-[(4-dimethylamino-cyclohexyl)amino]-pyrimido[5,4-d]pyrimidine,

(2) 4-[(R)-(1-phenylethyl)amino]-6-(4-hydroxyphenyl)-7H-pyrrolo[2,3-d]pyrimidine,

(3) 4-{[3-chloro-4-(3-fluoro-4-benzyloxy)-phenyl]amino}-6-(5-{[(2-methanesulphonyl-ethyl)amino]methyl}-furan-2-yl)quinazoline or

the antibody Cetuximab C225, Trastuzumab, ABX-EGF, Mab ICR-62 or EGFR-antisense,

the tautomers, the stereoisomers or the salts thereof.

A particularly preferred object of the invention is the use of the compounds of general formula (I) wherein

X denotes a nitrogen atom or a carbon atom substituted by a cyano group,

R^{a} denotes a hydrogen atom,

R^{b} denotes a phenyl or 1-phenylethyl group, wherein the phenyl nucleus in each case is substituted by the groups R¹ and R², while

R¹ and R², which may be identical or different, in each case denote a hydrogen, fluorine, chlorine or bromine atom,

a C₁₋₄-alkyl, C₂₋₅-alkenyl or C₂₋₅-alkynyl group,

A denotes an oxygen atom or an imino group,

B denotes a bond or a carbonyl group,

C denotes a methylene, ethylene or ethenylene group,

n denotes one of the numbers 0 or 1,

D denotes a di-(C₁₋₄-alkyl)-amino group wherein the alkyl moieties may be identical or different,

a methylamino or ethylamino group, wherein in each case the nitrogen atom is substituted by a 2-methoxyethyl, tetrahydrofuran-3-yl, tetrahydropyran-4-yl, tetrahydrofuran-2-ylmethyl, cyclopropyl or cyclopropylmethyl group,

an N—(C₁₋₄-alkyl)—N—(C₂₋₄-alkyl)-amino group wherein the alkyl moieties in the β, γ or δ position to the nitrogen atom of the amino group may optionally be substituted by the group R³, while

R³ denotes a C₁₋₃-alkoxy or C₁₋₃-alkoxycarbonyl group,

a bis-(2-methoxyethyl)-amino group,

a morpholino or 2-oxo-morpholin-4-yl group optionally substituted by a methyl or methoxymethyl group,

a hydroxy or C₁₋₄-alkoxy group, or also

a hydrogen atom, if n is the number 0, and

R^{c} denotes a hydrogen atom, a C₁₋₄-alkoxy-C₁₋₄-alkoxy, C₁₋₄-alkoxy, C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group,

a 3-pyrrolidinyloxy, 2-pyrrolidinyl-C₁₋₃-alkyloxy, 3-pyrrolidinyl-C₁₋₃-alkyloxy, 3-piperidinyloxy, 4-piperidinyloxy, 2-piperidinyl-C₁₋₃-alkyloxy, 3-piperidinyl-C₁₋₃-alkyloxy or 4-piperidinyl-C₁₋₃-alkyloxy group, wherein in each case the cyclic nitrogen atom is substituted by the group R⁴, where R⁴ is as hereinbefore defined,

a piperazino or homopiperazino group substituted in the 4 position by an R⁶—C₁₋₄-alkyl, R⁶—CO or R⁶—C₁₋₄-alkylene-CO group, wherein

R⁶ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups,

a morpholino-C₁₋₄-alkoxy or 2-oxo-morpholin-4-yl-C₁₋₆-alkoxy group which may be substituted by 1 or 2 methyl or ethyl groups, or

a tetrahydrofuran-3-yloxy, tetrahydropyran-3-yloxy, tetrahydropyran-4-yloxy, tetrahydrofuranylmethoxy or tetrahydropyranylmethoxy group, or

the compounds

(1) 4-[(3-chloro-4-fluorophenyl)amino]-6-[(4-dimethylamino-cyclohexyl)amino]-pyrimido[5,4-d]pyrimidine,

(2) 4-[(R)-(1-phenylethyl)amino]-6-(4-hydroxyphenyl)-7H-pyrrolo[2,3-d]pyrimidine,

(3) 4-{[3-chloro-4-(3-fluoro-4-benzyloxy)-phenyl]amino}-6-(5-{[(2-methanesulphonyl-ethyl)amino]methyl}-furan-2-yl)quinazoline or

the antibody Cetuximab C225, Trastuzumab, ABX-EGF, Mab ICR-62 or EGFR-antisense,

the tautomers, the stereoisomers or the salts thereof.

The following compounds of general formula (I) may be used, for example, for the purpose according to the invention:

(1) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,

(2) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,

(3) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,

(4) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,

(5) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline

(6) 4-[(3-chloro-4-fluorophenyl)amino]-6-{[4-(morpholin-4-yl)-1-oxo-2-buten-1-yl]-amino}-7-cyclopropylmethoxy-quinazoline,

(7) 4-[(3-chloro-4-fluorophenyl)amino]-6-{[4-(N,N-diethylamino)-1-oxo-2-buten-yl]amino}-7-cyclopropylmethoxy-quinazoline,

(8) 4-[(3-chloro-4-fluorophenyl)amino]-6-{[4-(N,N-dimethylamino)-1-oxo-2-buten-1-yl]amino}-7-cyclopropylmethoxy-quinazoline,

(9) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,

(10) 4-[(R)-(1-phenyl-ethyl)amino]-6-{[4-(morpholin-4-yl)-1-oxo-2-buten-1-yl]amino}-7-cyclopropylmethoxy-quinazoline,

(11) 4-[(R)-(1-phenyl-ethyl)amino]-6-{[4-(morpholin-4-yl)-1-oxo-2-buten-1-yl]amino}-7-cyclopentyloxy-quinazoline,

(12) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,

(13) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline

(14) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline,

(15) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,

(16) 4-[(3-chloro-4-fluoro-phenyl)amino]-6-[2-((S)-6-methyl-2-oxo-morpholin-4-yl)-ethoxy]-7-methoxy-quinazoline,

(17) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,

(18) 4-[(3-chloro-4-fluorophenyl)amino]-6-{[4-(N,N-dimethylamino)-1-oxo-2-buten-1-yl]amino}-7-cyclopentyloxy-quinazoline,

(19) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline

(20) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,

(21) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,

(22) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,

(23) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,

(24) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,

(25) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,

(26) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,

(27) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,

(28) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,

(29) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,

(30) 4-[(3-chloro-4-fluoro-phenyl)amino]-6-[(4-dimethylamino-cyclohexyl)amino]-pyrimido[5,4-d]pyrimidine

(31) 4-[(3-chloro-4-fluorophenyl)amino]-6-[3-(morpholin-4-yl)-propyloxy]-7-methoxy-quinazoline,

(32) 4-[(3-ethynyl-phenyl)amino]-6,7-bis-(2-methoxy-ethoxy)-quinazoline,

(33) 4-[(3-chloro-4-fluorophenyl)amino]-7-[3-(morpholin-4-yl)-propyloxy]-6-[(vinyl-carbonyl)amino]-quinazoline,

(34) 4-[(R)-(1-phenyl-ethyl)amino]-6-(4-hydroxy-phenyl)-7H-pyrrolo[2,3-d]pyrimidine,

(35) 3-cyano-4-[(3-chloro-4-fluorophenyl)amino]-6-{[4-(N,N-dimethylamino)-1-oxo-2-buten-1-yl]amino}-7-ethoxy-quinoline,

(36) 4-{[3-chloro-4-(3-fluoro-benzyloxy)-phenyl]amino}-6-(5-{[(2-methanesulphonyl-ethyl)amino]methyl}-furan-2-yl)quinazoline,

(37) Cetuximab,

(38) Trastuzumab,

(39) ABX-EGF,

(40) Mab ICR-62,

(41) EGFR-antisense

or their salts, while

the compounds

(1) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,

(2) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,

(3) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,

(4) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,

(5) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline

(6) 4-[(3-chloro-4-fluorophenyl)amino]-6-{[4-(morpholin-4-yl)-1-oxo-2-buten -1-yl]-amino}-7-cyclopropylmethoxy-quinazoline,

(7) 4-[(3-chloro-4-fluorophenyl)amino]-6-{[4-(N,N-diethylamino)-1-oxo-2-buten-1-yl]amino}-7-cyclopropylmethoxy-quinazoline,

(8) 4-[(3-chloro-4-fluorophenyl)amino]-6-{[4-(N,N-dimethylamino)-1-oxo-2-buten-1-yl]amino}-7-cyclopropylmethoxy-quinazoline,

(9) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,

(10) 4-[(R)-(1-phenyl-ethyl)amino]-6-{[4-(morpholin-4-yl)-1-oxo-2-buten-1-yl]amino}-7-cyclopropylmethoxy-quinazoline,

(11) 4-[(R)-(1-phenyl-ethyl)amino]-6-{[4-(morpholin-4-yl)-1-oxo-2-buten-1-yl]amino}-7-cyclopentyloxy-quinazoline,

(12) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,

(13) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline,

(14) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline,

(15) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,

(16) 4-[(3-chloro-4-fluoro-phenyl)amino]-6-[2-((S)-6-methyl-2-oxo-morpholin-4-yl)-ethoxy]-7-methoxy-quinazoline,

(17) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,

(18) 4-[(3-chloro-4-fluorophenyl)amino]-6-{[4-(N,N-dimethylamino)-1-oxo-2-buten-1-yl]amino}-7-cyclopentyloxy-quinazoline,

(19) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,

(20) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,

(21) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,

(22) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,

(23) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,

(24) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,

(25) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,

(26) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,

(27) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,

(28) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,

(29) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,

(30) 4-[(3-chloro-4-fluoro-phenyl)amino]-6-[(4-dimethylamino-cyclohexyl)amino]-pyrimido[5,4-d]pyrimidine,

(31) 4-[(3-chloro-4-fluorophenyl)amino]-6-[3-(morpholin-4-yl)-propyloxy]-7-methoxy-quinazoline,

or their salts are to be regarded as preferred and

the compounds

(1) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,

(2) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,

(3) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,

(4) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,

(5) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,

(6) 4-[(R)-(1-phenyl-ethyl)amino]-6-{[4-(morpholin-4-yl)-1-oxo-2-buten-1-yl]amino}-7-cyclopropylmethoxy-quinazoline and

(7) 4-[(3-chloro-4-fluorophenyl)amino]-6-[3-(morpholin-4-yl)-propyloxy]-7-methoxy-quinazoline

or their salts are to be regarded as particularly preferred.

The present invention further relates to a process for the treatment of

diseases of the airways or lungs which are accompanied by increased or altered production of mucus, such as e.g. inflammatory diseases of the airways such as acute bronchitis, chronic bronchitis, chronic obstructive bronchitis (COPD), asthma, bronchiectasis, allergic or non-allergic rhinitis or sinusitis, cystic fibrosis, α1-antitrypsin deficiency, or coughs, lung emphysema, pulmonary fibrosis and hyperreactive airways,

for the treatment of inflammatory diseases of the gastrointestinal tract and the bile duct and gall bladder which are accompanied by impaired tyrosine kinase function, such as may be found for example in acute or chronic inflammatory changes such as cholecystitis, Crohn's disease, ulcerative colitis, as well as ulcers and polyposis in the gastro-intestinal tract or such as occur in diseases of the gastro-intestinal tract which are associated with increased secretion, such as Ménétrier's disease, secreting adenomas and protein loss syndrome,

and also for the treatment of inflammatory diseases of the joints, such as rheumatoid arthritis, inflammatory diseases of the skin and the eyes, inflammatory pseudopolyps, as well as colitis cystica profunda and pneumatosis cystoides intestinalis. comprising administering an effective amount of one or more of the abovementioned compounds of general formula (I) according to the invention or optionally one of the physiologically acceptable salts thereof to a patient requiring such treatment.

Preferred and particularly preferred embodiments of the process according to the invention correspond to the embodiments mentioned above for use according to the invention, in terms of the particular compounds and indications.

In the process according to the invention the abovementioned compounds are used in dosages of 0.001-10 mg/kg of body weight, preferably 0.01-1.5 mg/kg, conveniently administered 1 to 3 times a day.

The active substances may be administered by oral, buccal or parenteral route, by inhaling sprays, or by rectal or topical application. They may be administered parenterally by subcutaneous, intravenous and intramuscular injections and infusion techniques.

For this purpose, conventional formulations may be used, such as the preparations mentioned hereinbefore for the active substances. For example, the active substances, optionally combined with other active substances, may be formulated with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The active substances may be administered orally in a wide variety of different dosage forms, for example they may be formulated together with different pharmaceutically acceptable inert carriers in the form of tablets, capsules, pastilles, lozenges, hard sweets, powders, atomisers, aqueous suspensions, elixirs, syrups and the like. Such carriers include for example solid diluents or fillers, sterile aqueous media and various non-toxic organic solvents. In addition, oral formulations of this kind may be suitably sweetened and/or flavoured with various agents conventionally used for this purpose. In general, the active substances are present in oral formulations of this kind at concentration levels ranging from about 0.5 wt. % to about 90 wt. %, based on the total composition, in amounts sufficient to produce the desired dosage units. Other suitable dosage forms for the active substances comprise formulations for controlled release and devices which are well known to the specialists in the field.

For the purposes of parenteral administration, solutions of the active substances in sesame or groundnut oil or in aqueous propyleneglycol may be used, as well as sterile aqueous solutions of the corresponding pharmaceutically acceptable salts. Such aqueous solutions should if necessary be suitably buffered and the liquid diluent made isotonic with sufficient salt or glucose. These specific aqueous solutions are particularly suitable for intravenous, intramuscular and subcutaneous injections. In connection with this, the sterile aqueous media used may easily be obtained using common methods well known in the art. For example, distilled water is normally used as the liquid diluent, and the final preparation is passed through a suitable bacterial filter such as a filter made of sintered glass or kieselguhr or unglazed porcelain. Preferred filters of this kind include the Berkefeld, Chamberland and asbestos disc metal Seitz filter, in which the fluid is sucked into a sterile container by means of a suction pump. During the preparation of these injectable solutions the necessary process steps should be taken at all times to ensure that the end products are obtained in a sterile condition. For the purposes of transdermal administration, the dosage form of the particular compound or compounds may comprise, for example, solutions, lotions, ointments, creams, gels, suppositories, formulations for continuous speed-limited release and equipment for this purpose. Such dosage forms comprise the particular compound or compounds and may contain ethanol, water, penetration promoters and inert carriers such as gel producers, mineral oil, emulsifiers, benzylalcohol and the like.

The compounds are administered by inhalation in the form of powdered preparations with lactose and other excipients or in the form of aqueous solutions as aerosols.

The inhalable powders which may be used within the scope of the invention may contain the active substance or combination of active substances either on their own or in admixture with suitable physiologically acceptable excipients. If the active substance or combination of active substances is present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose, particularly but not exclusively in the form of the hydrates thereof, is preferred. Lactose is particularly preferred, while lactose monohydrate is most preferred, as the excipient according to the invention.

The propellant-containing aerosols for inhalation which may be used within the scope of the use according to the invention may contain the active substance or combination of active substances dissolved in the propellent gas or in dispersed form. The propellent gases which may be used to prepare the aerosols for inhalation are known from the prior art. Suitable propellent gases are selected from among the hydrocarbons such as n-propane, n-butane or isobutane and halohydrocarbons such as preferably fluorinated derivatives of methane, ethane, propane, butane, cyclopropane or cyclobutane. The abovementioned propellent gases may be used on their own or mixed together. Particularly preferred propellent gases are fluorinated alkane derivatives selected from TG134a (1,1,1,2-tetrafluoroethane), TG227 (1,1,1,2,3,3,3-heptafluoropropane) and mixtures thereof.

The propellant-containing aerosols for inhalation which may be used within the scope of the use according to the invention may further contain additional ingredients such as cosolvents, stabilisers, surfactants, antioxidants, lubricants as well as pH adjusters. All these ingredients are known in the art.

If the active substance or combination of active substances according to the invention is administered by inhalation in the form of propellant-free solutions or suspensions, aqueous or alcoholic, preferably ethanolic solutions may be used as solvent. The solvent may be exclusively water or it may be a mixture of water and ethanol. The relative proportion of ethanol to water is not restricted, but the maximum limit is preferably up to 70 percent by volume, particularly up to 60 percent by volume and most particularly up to 30 percent by volume. The remaining percent by volume are made up of water. Solutions or suspensions containing the active substance or combination of active substances are optionally adjusted with suitable acids to a pH of 2 to 7, preferably 2 to 5. This pH may be adjusted using acids selected from inorganic or organic acids. Examples of particularly suitable inorganic acids are hydrochloric acid, hydrobromic acid, nitric acid, sulphuric acid and/or phosphoric acid. Examples of particularly suitable organic acids are: ascorbic acid, citric acid, malic acid, tartaric acid, maleic acid, succinic acid, fumaric acid, acetic acid, formic acid and/or propionic acid and others. Preferred inorganic acids are hydrochloric acid, sulphuric acid. Of the organic acids ascorbic acid, fumaric acid and citric acid are preferred. If desired, mixtures of the abovementioned acids may also be used, particularly in the case of acids which have other properties, in addition to their acidifying properties, e.g. as flavourings, antioxidants or complexing agents, such as for example citric acid or ascorbic acid. According to the invention, hydrochloric acid is most preferably used to adjust the pH.

As already mentioned at the beginning, the compounds of general formula (I) and their salts have valuable properties, particularly an anti-inflammatory activity.

For example the compounds

A=4-[(3-chloro-4-fluoro-phenyl)amino]-6-[2-((S)-6-methyl-2-oxo-morpholin-4-yl)-ethoxy]-7-methoxy-quinazoline,

B=4-[(3-chloro-4-fluoro-phenyl)amino]-7-[4-((R)-6-methyl-2-oxo-morpholin-4-yl)-butyloxy]-6-[(vinylcarbonyl)amino]-quinazoline,

C=4-[(3-chloro-4-fluoro-phenyl)amino]-7-[4-(2,2-dimethyl-6-oxo-morpholin-4-yl)-butyloxy]-6-[(vinylcarbonyl)amino]-quinazoline,

D=4-[(3-chloro-4-fluoro-phenyl)amino]-7-[4-((S)-6-methyl-2-oxo-morpholin-4-yl)-butyloxy]-6-[(vinylcarbonyl)amino]-quinazoline,

E=4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline,

F=4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,

G=4-[(3-chloro-4-fluoro-phenyl)amino]-6-[2-((S)-6-methyl-2-oxo-morpholin-4-yl)-ethoxy]-7-methoxy-quinazoline,

H=4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,

I=4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,

K=4-[(R)-(1-phenyl-ethyl)amino]-6-{[4-(N,N-bis-(2-methoxy-ethyl)-amino)-1-oxo-2buten-1-yl]amino}-7-cyclopropylmethoxy-quinazoline,

L=4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline and

M=4-[(3-chloro-4-fluorophenyl)amino]-6-[3-(morpholin-4-yl)-propyloxy]-7-methoxy-quinazoline

were subjected to the following tests to investigate their anti-inflammatory activity:

Test 1: Inhibition of smoke-induced accumulation of granulocytes in the lung tissue

Lung indications: Inhibition of cigarette smoke-induced influx of neutrophilic granulocytes into the lung tissue by the EGF receptor kinase inhibitor 4-[(3-chloro-4-fluoro-phenyl)amino]-6-[2-((S)-6-methyl-2-oxo-morpholin-4-yl)-ethoxy]-7-methoxy-quinazoline.

Method:

Male rats (breed: Sprague-Dawley) weighing from 250-300 g were exposed to the smoke from 8 cigarettes a day for 5 days. The animals in the group treated with 4-[(3-chloro-4-fluoro-phenyl)amino]-6-[2-((S)-6-methyl-2-oxo-morpholin-4-yl)-ethoxy]-7-methoxy-quinazoline (compound A) were given an intratracheal dose of 0.03 or 0.1 mg/kg of compound A in a volume of 0.05 ml each day, 30 mins before the start of the smoke exposure, while anaesthetised with isofluran. On the last day of the experiment the animals were killed 4 hours after the final smoke exposure and the lung tissue was removed. From each lung a sample of 70-200 mg was taken and placed in a prepared test tube containing 1 ml of 0.5% hexadecyltrimethyl ammonium bromide. The samples were homogenised for 15 sec with an Ultraturrax. The homogenates were centrifuged off in an Eppendorf bench centrifuge at 15700 g for 5 min at ambient temperature. 50 ml were taken from the supernatant and mixed with 250 ml of phosphate buffer (50 mmol/1) containing 0.197 mg/ml of O-dianisidine dihydrochloride. After 10 minutes' incubation at ambient temperature, the absorption was measured with a spectral photometer at a wavelength of 450 nm.

The dosage that produced a 50% inhibition of the MPO activity (=ID50) was determined by linear regression.

Results:

Exposure to cigarette smoke led to an influx of neutrophilic granulocytes into the lung tissue in rats, measured by the tissue content of myeloperoxidase, which is specific for neutrophilic granulocytes. Intratracheal treatment of the animals with the EGFR kinase inhibitor A resulted in a significant (p<0.005) inhibition of the smoke-induced accumulation of granulocytes and thus produced an anti-inflammatory activity.

Further results are shown in the following Table:

| active substance | ID50 [mg/kg] |
|---|---|
| A | 0.1 |
| B | 0.03 |
| C | 0.03 |
| D | 0.3 |
| E | 0.2 |
| F | 0.3 |
| G | <0.03 |
| H | 0.3 |
| I | 0.2 |
| K | 0.3 |
| L | 0.1 |
| M | 0.30 |

Test 2: Detection of a general anti-inflammatory principle of activity by inhibition of the zymosan-induced influx of neutrophilic granulocytes in the mouse ear by the EGF-receptor kinase inhibitor 4-[(3-chloro-4-fluoro-phenyl)amino]-6-[(4-dimethylamino-cyclohexyl)amino]-pyrimido[5,4-d]pyrimidine (compound N).

Method:

Determining the influx of neutrophilic granulocytes by measuring the myeloperoxidase (MPO) activity in the tissue. MPO is specific for neutrophilic granulocytes. Female mice (breed: NMRI) weighing 20-25 g were anaesthetised with pentobarbital 60 mg/kg i.p.. 10 {cube root}g of zymosan dissolved in physiological saline in a volume of 10 □l were administered intradermally into the right ear. 24 h after the intradermal application of zymosan the animals were killed with an overdose of pentobarbital. An ear biopsy (=8 mm) was taken from the left (untreated) and right (treated) ear and placed in a test tube prepared with 1 ml of 0.5% HTAB. The samples were homogenised for 15 sec with an Ultraturrax. The homogenised preparations were centrifuged for 5 min in an Eppendorf bench centrifuge at 15700 g at ambient temperature. 50 ml were taken from the supernatant and mixed with 250 ml of phosphate buffer (50 mmol/l), containing 0.197 mg/ml of O-dianisidine dihydrochloride. After 10 minutes' incubation at ambient temperature the absorption was measured with a spectral photometer at a wavelength of 450 nm.

Results:

The intradermal injection of zymosan led to a significant increase in the MPO activity in the tissue. Treating the animals with the EGFR kinase inhibitor 4-[(3-chloro-4-fluoro-phenyl)amino]-6-[(4-dimethylamino-cyclohexyl)amino]-pyrimido[5,4-d]pyrimidine inhibited this increase significantly (p<0.005) by 60%.

The abovementioned compounds, the preparation of which is not already in the art, are obtained by the following methods:

### EXAMPLE 1

4-[(3-chloro-4-fluoro-phenyl)amino]-7-{3-[4-(2-oxo-tetrahydrofuran-4-yl)-piperazin-1-yl]-propyloxy}-6-[(vinylcarbonyl)amino]-quinazoline

A mixture of 166 mg of acrylic acid and 0.77 ml of triethylamine in 10 ml of tetrahydrofuran is cooled to −50° C. in a dry ice/acetone cooling bath and combined with a solution of 175 μl of acrylic acid chloride in 4 ml of tetrahydrofuran. The reaction mixture is stirred for 45 minutes at this temperature. Then a solution of 427 mg 6-amino-4-[(3-chloro-4-fluoro-phenyl)amino]-7-{3-[4-(2-oxo-tetrahydrofuran-4-yl)-piperazin-1-yl]-propyloxy}-quinazoline in 10 ml of tetrahydrofuran is added dropwise within 20 minutes.

The reaction mixture is then slowly allowed to come up to 0° C. and stirred at this temperature until the reaction is complete. Then ice water is added, whereupon a viscous precipitate is formed. This is extracted thoroughly several times with ethyl acetate/methanol. The combined organic phases are washed with saturated sodium chloride solution, dried over magnesium sulphate and evaporated down. The yellowish, resinous crude product is purified by chromatography over a silica gel column with methylene chloride/methanol (95:5) as eluant.

Yield: 148 mg (31% of theory),

R_{f} value: 0.45 (silica gel, methylene chloride/methanol/concentrated, aqueous ammonia solution=90: 10:0.1)

Mass spectrum (ESI): m/z=567, 569 [M−H]

The following compound is obtained analogously to Example 1:

4-[(3-chloro-4-fluoro-phenyl)amino]-7-(2-{4-[(S)-(2-oxo-tetrahydrofuran-5-yl)carbonyl]piperazin-1-yl}-ethoxy)-6-[(vinylcarbonyl)amino]-quinazoline

R_{f} value: 0.46 (silica gel, methylene chloride/methanol/concentrated aqueous ammonia solution=90:10:0.1)

Mass spectrum (ESI): m/z=581, 583 [M−H]

### EXAMPLE 2

4-[(3-chloro-4-fluoro-phenyl)amino]-7-[3-(2,2-dimethyl-6-oxo-morpholin-4-yl)-propyloxy]-6-[(vinylcarbonyl)amino]-quinazoline

0.47 ml of triethylamine are added to 101 mg of acrylic acid in 5 ml of tetrahydrofuran under a nitrogen atmosphere. This mixture is cooled to about −50° C. in a dry ice/acetone cooling bath and combined with 119 mg acrylic acid chloride in 3 ml of tetrahydrofuran, whereupon a colourless precipitate is formed. The suspension is stirred for about another hour at this temperature. Then 240 mg 6-amino-4-[(3-chloro-4-fluoro-phenyl)amino]-7-[3-(2,2-dimethyl-6-oxo-morpholin-4-yl)-propyloxy]-quinazoline in 7 ml of tetrahydrofuran are added dropwise at −55° C. The reaction mixture is allowed to warm up slowly to −30° C. in the cooling bath. After about an hour the dry ice/acetone cooling bath is replaced by an ice/sodium chloride cooling bath. The reaction mixture is allowed to warm up to 0° C. therein. As soon as the reaction is complete, the reaction mixture is combined with water and methylene chloride and made alkaline with sodium hydroxide solution. The aqueous phase separated off is extracted again with methylene chloride and a little methanol. The combined organic extracts are washed with water, dried and evaporated down. A yellow resin remains which is chromatographed through a silica gel column with methylene chloride/methanol (98:2) as eluant. The desired product is stirred with a little tert.butylmethyl ether, the fine crystalline precipitate is suction filtered, washed with tert.butylmethyl ether and dried at 50° C. in vacuo.

Yield: 160 mg (60% of theory),

R_{f} value: 0.42 (silica gel, methylene chloride/methanol=95:5)

Mass spectrum (ESI): m/z=526, 528 [M−H]

The following compounds are obtained analogously to Example 2:

(1) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline

R_{f} value: 0.32 (silica gel, methylene chloride/methanol=95:5)

Mass spectrum (ESI): m/z=498, 500 [M−H]

(2) 4-[(3-chloro-4-fluoro-phenyl)amino]-7-[4-((R)-6-methyl-2-oxo-morpholin-4-yl)-butyl-oxy]-6-[(vinylcarbonyl)amino]-quinazoline

R_{f} value: 0.30 (silica gel, methylene chloride/methanol=95:5)

Mass spectrum (ESI): m/z=550, 552 [M+Na]

(3) 4-[(3-chloro-4-fluoro-phenyl)amino]-7-[4-((S)-6-methyl-2-oxo-morpholin-4-yl)-butyl-oxy]-6-[(vinylcarbonyl)amino]-quinazoline

Mass spectrum (ESI): m/z=526, 528 [M−H]

(4) 4-[(3-chloro-4-fluoro-phenyl)amino]-7-[4-(2,2-dimethyl-6-oxo-morpholin-4-yl)-butyl-oxy]-6-[(vinylcarbonyl)amino]-quinazoline

melting point: 110-112° C.

Mass spectrum (ESI⁻): m/z=540, 542 [M−H]⁻

### EXAMPLE 3

4-[(3-chloro-4-fluorophenyl)amino]-6-{[4-(N,N-diethylamino)-1-oxo-2-buten-1-yl]amino}-7-cyclopropylmethoxy-quinazoline

To a solution of 640 mg 4-bromo-2-butenoic acid in 10 ml methylene chloride are added at ambient temperature 0.67 ml oxalyl chloride and one drop of dimethyl formamide. The reaction mixture is stirred for roughly another half hour at ambient temperature until the development of gas has ended. The acid chloride formed is largely freed from solvent in vacuo using the rotary evaporator. Then the crude product is dissolved in 10 ml methylene chloride and while cooling with an ice bath added dropwise to a mixture of 1.00 g 6-amino-4-[(3-chloro-4-fluorophenyl)amino]-7-cyclopropylmethoxy-quinazoline and 1.60 ml Hünig base in 50 ml of tetrahydrofuran. The reaction mixture is stirred for 1.5 hours in the ice bath and for a further 2 hours at ambient temperature. Then 2.90 ml diethylamine are added and the mixture is stirred for 2.5 days at ambient temperature. For working up the reaction mixture is filtered and the filtrate is evaporated down. The flask residue is purified by chromatography over a silica gel column with ethyl acetate/methanol (19:1).

Yield: 550 mg (40% of theory)

Melting point: 114° C.

Mass spectrum (ESI): m/z=498, 500 [M+H]

The following compounds are obtained analogously to Example 3:

(1) 4-[(3-chloro-4-fluoro-phenyl)amino]-6-{[4-(morpholin-4-yl)-1-oxo-2-buten-1-yl]-amino}-7-cyclopropylmethoxy-quinazoline

R_{f} value: 0.53 (silica gel, ethyl acetate/methanol=9:1)

Mass spectrum (ESI): m/z=510, 512 [M−H]

(2) 4-[(3-chloro-4-fluorophenyl)amino]-6-{[4-(N,N-dimethylamino)-1-oxo-2-buten-1-yl]amino}-7-cyclopropylmethoxy-quinazoline

Melting point: 137° C.

Mass spectrum (ESI): m/z=470, 472 [M+H]

(3) 4-[(R)-(1-phenyl-ethyl)amino]-6-{[4-(morpholin4-yl)-1-oxo-2-buten-1-yl]amino}-7-cyclopropylmethoxy-quinazoline

R_{f} value: 0.37 (silica gel, ethyl acetate/methanol=9:1)

Mass spectrum (ESI): m/z=488 [M+H]

(4) 4-[(R)-(1-phenyl-ethyl)amino]-6-{[4-(morpholin-4-yl)-1-oxo-2-buten-1-yl]-amino}-7-cyclopentyloxy-quinazoline

R_{f} value: 0.35 (silica gel, ethyl acetate/methanol=9:1)

Mass spectrum (ESI): m/z=502 [M+H]

(5) 4-[(3-chloro-4-fluorophenyl)amino]-6-({4-[bis-(2-methoxyethyl)-amino]-1-oxo-2-buten-1-yl}amino)-7-cyclopropylmethoxy-quinazoline

R_{f} value: 0.51 (silica gel, ethyl acetate/methanol=9:1)

Mass spectrum (ESI⁺): m/z=558, 560 [M+H]⁺

### EXAMPLE 4

4-[(3-methylphenyl)amino]-6-[(4-{N-[(ethoxycarbonyl)methyl]-N-methylamino}-1-oxo-2-buten-1-yl)amino]-7-methoxy-quinazoline

To a solution of 842 mg 4-bromo-2-butenoic acid in 15 ml methylene chloride are added at ambient temperature 0.86 ml oxalyl chloride and one drop of dimethylformamide. The reaction mixture is stirred for about another hour at ambient temperature until the development of gas has ended. The acid chloride formed is largely freed from solvent in vacuo using the rotary evaporator. Then the crude product is taken up in 10 ml of methylene chloride and while cooling with an ice bath added dropwise within five minutes to a mixture of 1.0 g 6-amino-4-[(3-methylphenyl)amino]-7-methoxy-quinazoline and 2.0 ml Hünig base in 50 ml of tetrahydrofuran. The reaction mixture is stirred for two hours while cooling with an ice bath and for a further two hours at ambient temperature. Then 6.7 ml Hünig base, 5.48 g sarcosine ethyl ester hydrochloride and 3 ml of dimethylfonnamide are added and the whole is stirred overnight at ambient temperature. For working up the reaction mixture is evaporated down in vacuo using the rotary evaporator and the flask residue is distributed between 75 ml ethyl acetate and 75 ml of water. The organic phase is washed with water and saturated sodium chloride solution, dried over magnesium sulphate and evaporated down. The crude product is purified by chromatography over a silica gel column with methylene chloride/methanol (20: 1).

Yield: 326 mg (20% of theory)

Melting point: 122-124° C.

Mass spectrum (ESI): m/z=464 [M+H]

The following compound is obtained analogously to Example 4:

4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropylmethoxy-quinazoline

R_{f} value: 0.62 (aluminium oxide, cyclohexane/ethyl acetate=1:1)

Mass spectrum (EI): m/z=627, 629 [M]

### EXAMPLE 5

4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline

950 mg of 4-[(3-chloro-4-fluoro-phenyl)amino]-6-[(4-{N-[(ethoxycarbonyl)methyl]-N-((R)-2-hydroxy-3-methoxy-propyl)-amino}-1-oxo-2-buten-1-yl)amino]-7-cyclopropylmethoxy-quinazoline and 195 μl methanesulphonic acid in 10 ml acetonitrile are refluxed for about four hours. For working up the reaction mixture is cooled in a bath of ice water, combined with 75 ml of ethyl acetate and 25 ml saturated sodium hydrogen carbonate solution and stirred vigorously for 10 minutes. The organic phase is separated off, washed with saturated sodium hydrogen carbonate solution and saturated sodium chloride solution and dried over magnesium sulphate. The solvent is distilled off in vacuo, leaving a brownish foam.

Yield: 610 mg (69% of theory),

R_{f} value: 0.55 (silica gel, methylene chloride/methanol=9:1)

Mass spectrum (ESI): m/z=570, 572 [M+H]

The following compound is obtained analogously to Example 5:

4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline

### EXAMPLE 6

4-[(3-chloro-4-fluoro-phenyl)amino]-6-{[4-((S)-6-methyl-2-oxo-morpholin-4-yl)-1-oxo-2-buten-1-yl]amino}-7-cyclopropylmethoxy-quinazoline

A mixture of 700 mg 4-[(3-chloro-4-fluoro-phenyl)amino]-6-[(4-{N-[(tert.butyl-oxycarbonyl)methyl]-N-((S)-2-hydroxy-prop-1-yl)-amino}-1-oxo-2-buten-1-yl)amino]-7-cyclopropylmethoxy-quinazoline and 228 mg p-toluenesulphonic acid-hydrate in 20 ml acetonitrile is refluxed for five hours. Then another 200 mg p-toluenesulphonic acid hydrate are added and again the mixture is refluxed for five hours. For working up the reaction mixture is evaporated to dryness. The flask residue is distributed between ethyl acetate and saturated sodium carbonate solution. The organic phase is separated off, washed with saturated sodium carbonate solution, water and saturated sodium chloride solution, dried over magnesium sulphate and evaporated down. The oily residue is brought to crystallisation by stirring with 15 ml diethyl ether.

Melting point: 173-175° C.

Mass spectrum (ESI): m/z=540, 542 [M+H]

The following compounds are obtained analogously to Example 6:

(1) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline

R_{f} value: 0.54 (silica gel, methylene chloride/methanol=9:1)

Mass spectrum (ESI): m/z=540, 542 [M+H]

(2) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline

(The reaction is carried out with methanesulphonic acid in acetonitrile)

R_{f} value: 0.38 (silica gel, methylene chloride/methanol=9:1)

Mass spectrum (ESI): m/z=556, 558 [M+H]

### EXAMPLE 7

4-[(3-bromo-phenyl)amino]-6-[2-((S)-6-methyl-2-oxo-morpholin-4-yl)-ethoxy]-7-methoxy-quinazoline

90 μl of methanesulphonic acid are added to 380 mg 4-[(3-bromo-phenyl)amino]-6-(2-{N-[(tert.butyloxycarbonyl)methyl]-N-((S)-2-hydroxy-propyl)-amino}-ethoxy)-7-methoxy-quinazoline in 8 ml acetonitrile. The reaction mixture is refluxed for about three hours, then another equivalent of methanesulphonic acid is added and refluxing is continued until the reaction is complete. For working up the reaction mixture is diluted with ethyl acetate and washed with saturated sodium hydrogen carbonate solution and saturated sodium chloride solution. The organic phase is dried over magnesium sulphate and evaporated down in vacuo. The flask residue is stirred with diethyl ether and suction filtered. The title compound is obtained as a white solid.

Yield: 280 mg (85% of theory),

Melting point: 190° C.

Mass spectrum (ESI): m/z=485, 487 [M−H]

The following compound is obtained analogously to Example 7:

4-[(3-chloro-4-fluoro-phenyl)amino]-6-[2-((S)-6-methyl-2-oxo-morpholin-4-yl)-ethoxy]-7-methoxy-quinazoline

(The reaction is carried out with trifluoroacetic acid in acetonitrile)

Melting point: 212-213° C.

Mass spectrum (ESI): m/z=461, 463 [M+H]

### EXAMPLE 8

4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline

4.70 ml oxalyl chloride are added dropwise to a solution of 4.50 g bromocrotonic acid in 60 ml methylene chloride. Then one drop of N,N-dimethylformamide is added. After about 30 minutes the development of gas has ended and the reaction mixture is evaporated down in the rotary evaporator. The crude bromocrotonic acid chloride is taken up in 30 ml methylene chloride and while cooling with an ice bath added dropwise to a solution of 7.00 g 4-[(3-chloro-4-fluorophenyl)amino]-6-amino-7-cyclopropylmethoxy-quinazoline and 10.20 ml Hünig base in 150 ml of tetrahydrofuran. The reaction mixture is stirred for about 1.5 hours while cooling with an ice bath and for a further two hours at ambient temperature. 5.20 g of N-(2-methoxy-ethyl)-N-methyl-amine are then added and the reaction mixture is stirred overnight at ambient temperature. For working up it is diluted with methylene chloride and washed thoroughly with water. The organic phase is dried over magnesium sulphate and evaporated down. The crude product is purified by chromatography over a silica gel column with ethyl acetate followed by ethyl acetate/methanol (19:1) as eluant.

Yield: 5.07 g (51% of theory)

Mass spectrum (ESI): m/z=512, 514 [M−H]

R_{f} value: 0.25 (silica gel, ethyl acetate/methanol=9:1)

The following compounds are obtained analogously to Example 8:

(1) 4-[(3-chloro-4-fluorophenyl)amino]-6-{[4-(N,N-dimethylamino)-1-oxo-2-buten-1-yl]-amino}-7-cyclopentyloxy-quinazoline

Mass spectrum (ESI): m/z=482, 484 [M−H]

R_{f} value: 0.11 (silica gel, ethyl acetate/methanol=9:1)

(2) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline

Mass spectrum (ESI): m/z=532 [M−H]

R_{f} value: 0.40 (silica gel, ethyl acetate/methanol=9:1)

(3) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline

Mass spectrum (ESI): m/z=502 [M−H]

R_{f} value: 0.20 (silica gel, ethyl acetate/methanol=9:1)

(4) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline

Mass spectrum (ESI): m/z 488 [M−H]

R_{f} value: 0.25 (silica gel, ethyl acetate/methanol=9:1)

(5) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline

Mass spectrum (ESI): m/z=514 [M−H]

R_{f} value: 0.15 (silica gel, ethyl acetate/methanol=9:1)

(6) 4-[(3-chloro-4-fluorophenyl)amino]-6-{[4-(N,N-dimethylamino)-1-oxo-2-buten-1-yl]-amino}-7-((R)-tetrahydrofuran-3-yloxy)-quinazoline

Mass spectrum (ESI): m/z=486, 488 [M+H]

(7) 4-[(3-chloro-4-fluorophenyl)amino]-6-{[4-(N,N-dimethylamino)-1-oxo-2-buten-1-yl]-amino}-7-((S)-tetrahydrofuran-3-yloxy)-quinazoline

Mass spectrum (ESI): m/z=486, 488 [M+H]

R_{f} value: 0.45 (silica gel, methylene chloride/methanol=5:1)

(8) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline

Mass spectrum (ESI): m/z=528, 530 [M+H]

R_{f} value: 0.25 (silica gel, ethyl acetate/methanol=9:1)

(9) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline

Mass spectrum (ESI): m/z=508, 510 [M−H]

Melting point: 140° C.

(10) 4-[(3-chloro-4-fluorophenyl)amino]-6-{[4-(N,N-dimethylamino)-1-oxo-2-buten-1-yl]-amino}-7-[(R)-(tetrahydrofuran-2-yl)methoxy]-quinazoline

Mass spectrum (ESI): m/z=500, 502 [M+H]

Melting point: 110-112° C.

(11) 4-[(3-chloro-4-fluorophenyl)amino]-6-{[4-(N,N-dimethylamino)-1-oxo-2-buten-1-yl]-amino}-7-[(S)-(tetrahydrofuran-2-yl)methoxy]-quinazoline

Mass spectrum (ESI): m/z=500, 502 [M+H]

R_{f} value: 0.23 (silica gel, ethyl acetate/methanol/conc. aqueous ammonia=90:10:0.1)

Legend Relating to the Drawings

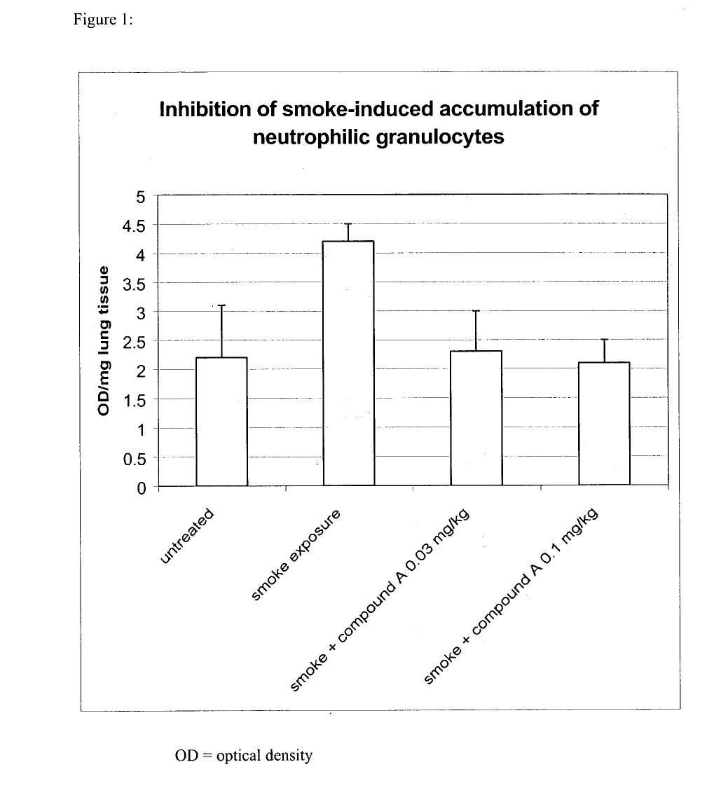

FIG. 1 shows the inhibition of the smoke-induced accumulation of neutrophilic granulocytes.

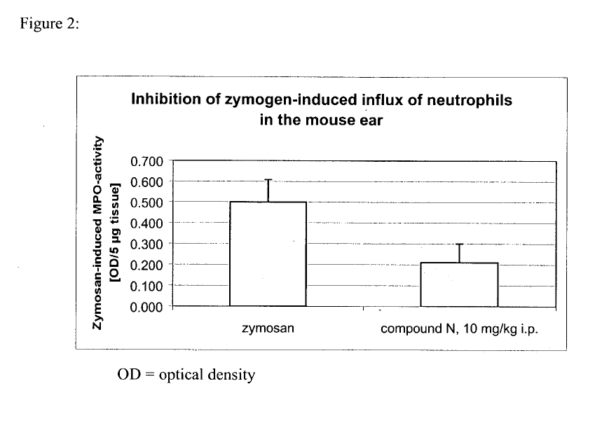

FIG. 2 shows the inhibition of the zymosan-induced influx of neutrophils in the mouse ear.

What is claimed is:

1. A method for treating a disease of the airways or lungs or the intestines associated with inflammation, which method comprises administering to a host in need of such treatment a therapeutically effective amount of a substance selected from the group consisting of:
(a) quinazolines of the formula 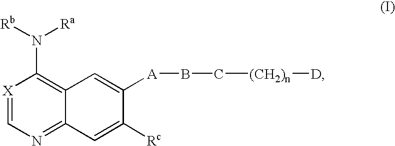
wherein
X denotes a nitrogen atom or a carbon atom substituted by a cyano group,
R^{a} denotes a hydrogen atom or a C₁₋₄-alkyl group,
R^{b} denotes a phenyl, benzyl or 1-phenylethyl group, wherein the phenyl nucleus may be substituted in each case by the groups R¹ and R², while
R¹ and R², which may be identical or different, in each case denote a hydrogen, fluorine, chlorine, bromine or iodine atom,
a C₁₋₄-alkyl, hydroxy, C₁₋₄-alkoxy, C₃₋₆-cycloalkyl, C₄₋₆-cycloalkoxy, C₂₋₅-alkeny or C₂₋₅-alkynyl group,
an aryl, aryloxy, arylmethyl or arylmethoxy group,
a C₃₋₅-alkenyloxy or C₃₋₅-alkynyloxy group, while the multiple bond is isolated from the oxygen atom,
a C₁₋₄-alkylsulphenyl, C₁₋₄-alkylsulphinyl, C₁₋₄-alkylsulphonyl, C₁₋₄-alkylsulphonyloxy, trifluoromethylsulphenyl, trifluoromethylsulphinyl or trifluoromethylsulphonyl group,
a methyl or methoxy group substituted by 1 to 3 fluorine atoms,
an ethyl or ethoxy group substituted by 1 to 5 fluorine atoms,
a cyano or nitro group or an amino group optionally substituted by one or two C₁₋₄-alkyl groups, while the substituents may be identical or different,
A denotes an oxygen atom or an imino group optionally substituted by a C₁₋₄-alkyl group,
B denotes a bond, a carbonyl or sulphonyl group,
C denotes a methylene, ethylene or ethenylene group,
n denotes one of the numbers 0 or 1,
D denotes an amino, C₁₋₄-alkylamino, C₃₋₅-cycloalkylamino or di-(C₁₋₄-alkyl)-amino or di-(C₃₋₅-cycloalkyl)-amino group wherein the alkyl and cycloalkyl moieties may be identical or different,
a C₂₋₄-alkylamino group wherein the alkyl moiety is substituted in the β, γ or δ position to the nitrogen atom of the amino group by the group R³, while
R³ denotes a hydroxy, C₁₋₄-alkoxy, C₁₋₃-alkoxycarbonyl, amino, C₁₋₄-alkylamino or di-(C₁₋₄-alkyl)-amino group,
a 4- to 7-membered alkyleneimino group optionally substituted by one or two methyl groups or
a 6- to 7-membered alkyleneimino group optionally substituted by one or two methyl groups wherein in each case a methylene group in the 4 position is replaced by an oxygen or sulphur atom, by a sulphinyl, sulphonyl, imino or N—(C₁₋₄-alkyl)-imino group,
an N—(C₁₋₄-alkyl)—N—(C₂₋₄-alkyl)-amino group wherein the alkyl moieties in the β, γ or δ position to the nitrogen atom of the amino group may optionally be substituted by the group R³, where R³ is as hereinbefore defined,
a di-(C₂₋₄-alkyl)-amino group wherein the two C₂₋₄-alkyl moieties in each case are substituted in the β, γ or δ position to the nitrogen atom of the amino group by the group R³, while the substituents may be identical or different and R³ is as hereinbefore defined,
a C₃₋₇-cycloalkylamino or C₃₋₇-cycloalkyl-C₁₋₃-alkylamino group, wherein in each case the nitrogen atom may be substituted by a further C₁₋₄-alkyl group, an amino or C₁₋₄-alkylamino group, wherein in each case the nitrogen atom is substituted by a tetrahydrofuran-3-yl, tetrahydropyran-3-yl, tetrahydropyran-4-yl, tetrahydrofuranylmethyl, 1-(tetrahydrofuran-3-yl)-piperidin-4-yl, 1-(tetrahydropyran-3-yl)-piperidin-4-yl, 1-(tetrahydropyran-4-yl)-piperidin-4-yl, 3-pyrrolidinyl, 3-piperidinyl, 4-piperidinyl, 3-hexahydro-azepinyl or 4-hexahydro-azepinyl group optionally substituted by 1 to 3 C₁₋₄-alkyl groups,
a 4- to 7-membered alkyleneimino group optionally substituted by 1 to 4 C₁₋₂-alkyl groups which may be substituted by the group R³ either at a cyclic carbon atom or at one of the alkyl groups, while R³ is as hereinbefore defined,
a piperidino group substituted by a tetrahydrofuranyl, tetrahydropyranyl or tetrahydrofuranylmethyl group,
a 6- to 7-membered alkyleneimino group optionally substituted by 1 or 2 C₁₋₂-alkyl groups wherein in each case a methylene group in the 4 position is replaced by an oxygen or sulphur atom, by an imino group substituted by the group R⁴, by a sulphinyl or sulphonyl group, while
R⁴ denotes a hydrogen atom, a C₁₋₄-alkyl, 2-methoxy-ethyl, 3-methoxy-propyl, C₃₋₇-cycloalkyl, C₃₋₇-cycloalkyl-C₁₋₄-alkyl, tetrahydrofuran-3-yl, tetrahydropyran-3-yl, tetrahydropyran-4-yl, tetrahydrofuranylmethyl, formyl, C₁₋₄-alkylcarbonyl, C₁₋₄-alkylsulphonyl, aminocarbonyl, C₁₋₄-alkylaminocarbonyl or di-(C₁₋₄-alkyl)-aminocarbonyl group,
a morpholino or 2-oxo-morpholin-4-yl group which may be substituted by a methyl, ethyl or C₁₋₃-alkoxymethyl group,
an imidazolyl group optionally substituted by 1 to 3 methyl groups,
a C₅₋₇-cycloalkyl group wherein a methylene group is replaced by an oxygen or sulphur atom, by an imino group substituted by the group R⁴, by a sulphinyl or sulphonyl group, while R⁴ is as hereinbefore defined,
a hydroxy or C₁₋₄-alkoxy group, or also
a hydrogen atom, if n is the number 0, and
R^{c} denotes a hydrogen atom, a C₁₋₄-alkoxy-C₁₋₄-alkoxy, C₁₋₄-alkoxy, C₄₋₇-cycloalkoxy or C₃₋₇-cycloalkyl-C₁₋₆-alkoxy group, wherein the cycloalkyl moiety may be substituted in each case by a C₁₋₃-alkyl, hydroxy, C₁₋₄-alkoxy, amino, C₁₋₄-alkylamino, di-(C₁₋₄-alkyl)-amino, pyrrolidino, piperidino, morpholino, piperazino, N—(C₁₋₂-alkyl)-piperazino,
hydroxy-C₁₋₂-alkyl, C₁₋₄-alkoxy-C₁₋₂-alkyl, amino-C₁₋₂-alkyl, C₁₋₄-alkylamino-C₁₋₂-alkyl, di-(C₁₋₄-alkyl)-amino-C₁₋₂-alkyl, pyrrolidino-C₁₋₂-alkyl, piperidino-C₁₋₂-alkyl, morpholino-C₁₋₂-alkyl, piperazino-C₁₋₂-alkyl or N—(C₁₋₂-alkyl)-piperazino-C₁₋₂-alkyl group, while the abovementioned monosubstituted cycloalkyl moieties may additionally be substituted by a C₁₋₃-alkyl group, or
a 3-pyrrolidinyloxy, 2-pyrrolidinyl-C₁₋₄-alkyloxy, 3-pyrrolidinyl-C₁₋₄-alkyloxy, 3-piperidinyloxy, 4-piperidinyloxy, 2-piperidinyl-C₁₋₄-alkyloxy, 3-piperidinyl-C₁₋₄-alkyloxy, 4-piperidinyl-C₁₋₄-alkyloxy, 3-hexahydro-azepinyloxy, 4-hexahydro-azepinyloxy, 2-hexahydro-azepinyl-C₁₋₄-alkyloxy, 3-hexahydro-azepinyl-C₁₋₄-alkyloxy or 4-hexahydro-azepinyl-C₁₋₄-alkyloxy group, wherein in each case the cyclic nitrogen atom is substituted by the group R⁴, where R⁴ is as hereinbefore defined,
a piperazino or homopiperazino group substituted in the 4 position by an R⁶-C₁₋₄-alkyl, R⁶—CO, R⁶-C₁₋₄-alkylene-CO, (R⁵NR⁷)-C₁₋₄-alkylene-CO, R⁷O—C₁₋₄-alkylene-CO, R⁷S—C₁₋₄-alkylene-CO, R⁷SO—C₁₋₄-alkylene-CO or R⁷SO₂-C₁₋₄-alkylene-CO group, wherein
R⁵ denotes a hydrogen atom or a C₁₋₄-alkyl group,
R⁶ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups and
R⁷ denotes a 2-oxo-tetrahydrofuran-3-yl, 2-oxo-tetrahydrofuran-4-yl, 2-oxo-tetrahydropyran-3-yl, 2-oxo-tetrahydropyran-4-yl or 2-oxo-tetrahydropyran-5-yl group optionally substituted by one or two C₁₋₂-alkyl groups,
a morpholino-C₁₋₄-alkoxy or 2-oxo-morpholin-4-yl-C₁₋₆-alkoxy group which may be substituted by 1 or 2 methyl or ethyl groups, or
a tetrahydrofuran-3-yloxy, tetrahydropyran-3-yloxy, tetrahydropyran-4-yloxy, tetrahydrofuranylmethoxy or tetrahydropyranylmethoxy group, while
by the aryl moieties mentioned in the definition of the abovementioned groups is meant a phenyl group, which may in each case be monosubstituted by R⁸, mono-, di- or trisubstituted by R⁹ or monosubstituted by R⁸ and additionally mono- or disubstituted by R⁹, while the substituents may be identical or different, wherein
R⁸ denotes a cyano, carboxy, C₁₋₄-alkoxycarbonyl, aminocarbonyl, C₁₋₄-alkyl-aminocarbonyl, di-(C₁₋₄-alkyl)-aminocarbonyl, C₁₋₄-alkylsulphenyl, C₁₋₄-alkylsulphinyl, C₁₋₄-alkylsulphonyl, hydroxy, C₁₋₄-alkylsulphonyloxy, trifluoromethyloxy, nitro, amino, C₁₋₄-alkylamino, di-(C₁₋₄-alkyl)-amino, C₁₋₄-alkylcarbonylamino, N—(C₁₋₄-alkyl)—C₁₋₄-alkylcarbonylamino, C₁₋₄-alkylsulphonylamino, N-(C₁₋₄-alkyl)-C₁₋₄-alkylsulphonylamino, aminosulphonyl, C₁₋₄-alkylaminosulphonyl or di-(C₁₋₄-alkyl)-aminosulphonyl group or a carbonyl group which is substituted by a 5- to 7-membered alkyleneimino group, while in the abovementioned 6- to 7-membered alkyleneimino groups in each case a methylene group in the 4 position may be replaced by an oxygen or sulphur atom, by a sulphinyl, sulphonyl, imino or N—(C₁₋₄-alkyl)-imino group, and
R⁹ denotes a fluorine, chlorine, bromine or iodine atom, a C₁₋₄-alkyl, trifluoromethyl or C₁₋₄-alkoxy group,
(b) the compounds
(1) 4-[(3-chloro-4-fluorophenyl)amino]-6-[(4-dimethylamino-cyclohexyl)amino]-pyrimido[5,4-d]pyrimidine,
(2) 4-[(R)-(1-phenylethyl)amino]-6-(4-hydroxyphenyl)-7H-pyrrolo[2,3-d]pyrimidine, and
(3) 4-{[3-chloro-4-(3-fluoro-4-benzyloxy)-phenyl]amino}-6-(5-{[(2-methanesulphonyl-ethyl)amino]methyl}-furan-2-yl)quinazoline,
as well as tautomers and pharmaceutically acceptable salts of one of the foregoing substances,
(c) the antibodies
(1) Cetuximab C225,
(2) Trastuzumab, ABX-EGF, and
(3) Mab ICR-62, and
(d) EGFR-antisense.

2. The method according to claim 1, wherein the substance administered is selected from the group consisting of:
(a) compounds of the formula I, wherein:
X denotes a nitrogen atom or a carbon atom substituted by a cyano group,
R^{a} denotes a hydrogen atom or a C₁₋₄-alkyl group,
R^{b} denotes a phenyl, benzyl or 1-phenylethyl group, wherein the phenyl nucleus may be substituted in each case by the groups R¹ and R², while
R¹ and R², which may be identical or different, in each case denote a hydrogen, fluorine, chlorine or bromine atom,
a C₁₋₄-alkyl, hydroxy, C₁₋₄-alkoxy, C₃₋₆-cycloalkyl, C₄₋₆-cycloalkoxy, C₂₋₅-alkeny or C₂₋₅-alkynyl group,
a methyl, trifluoromethyl or methoxy group,
A denotes an oxygen atom or an imino group optionally substituted by a C₁₋₄-alkyl group,
B denotes a bond or a carbonyl group,
C denotes a methylene, ethylene or ethenylene group,
n denotes one of the numbers 0 or 1,
D denotes a di-(C₁₋₄-alkyl)-amino group wherein the alkyl moieties may be identical or different,
an N—(C₁₋₄-alkyl)—N—(C₂₋₄-alkyl)-amino group wherein the alkyl moieties in the β, γ or δ position to the nitrogen atom of the amino group may optionally be substituted by the group R³, while
R³ denotes a hydroxy, C₁₋₃-alkoxy, C₁₋₃-alkoxycarbonyl, amino, C₁₋₄-alkylamino or di-(C₁₋₄-alkyl)-amino group,
a pyrrolidino, piperidino or morpholino group,
a di-(C₂₋₄-alkyl)-amino group wherein the two C₂₋₄-alkyl moieties are substituted in each case in the β, γ or δ position to the nitrogen atom of the amino group by the group R³, while the substituents may be identical or different and R³ is as hereinbefore defined,
a C₃₋₅-cycloalkylamino or C₃₋₅-cycloalkyl-C₁₋₃-alkylamino group, wherein in each case the nitrogen atom is substituted by a further C₁₋₄-alkyl group,
a C₁₋₄-alkylamino group wherein the nitrogen atom is substituted by a tetrahydrofuran-3-yl, tetrahydropyran-3-yl, tetrahydropyran-4-yl, tetrahydrofuranylmethyl, 1-(tetrahydrofuran-3-yl)-piperidin-4-yl, 1-(tetrahydropyran-3-yl)-piperidin-4-yl or 1-(tetrahydropyran-4-yl)-piperidin-4-yl group,
a 5- to 7-membered alkyleneimino group optionally substituted by 1 to 2 methyl groups which may be substituted by the group R³ either at a cyclic carbon atom or at one of the methyl groups, while R³ is as hereinbefore defined,
a piperidino group substituted by a tetrahydrofuranyl, tetrahydropyranyl or tetrahydrofuranylmethyl group,
a piperidino group optionally substituted by 1 or 2 methyl groups wherein the methylene group in the 4 position is replaced by an oxygen or sulphur atom, by an imino group substituted by the group R⁴, by a sulphinyl or sulphonyl group, while
R⁴ denotes a hydrogen atom, a C₁₋₃-alkyl, 2-methoxy-ethyl, 3-methoxy-propyl, C₃₋₆-cycloalkyl, C₃₋₆-cycloalkyl-C₁₋₃-alkyl, tetrahydrofuran-3-yl, tetrahydropyran-3-yl, tetrahydropyran-4-yl, tetrahydrofuranylmethyl, C₁₋₃-alkylcarbonyl, C₁₋₃-alkylsulphonyl, aminocarbonyl, C₁₋₃-alkylaminocarbonyl or di-(C₁₋₃-alkyl)-aminocarbonyl group,
a morpholino or 2-oxo-morpholin-4-yl group which may be substituted by a methyl, ethyl or C₁₋₃-alkoxymethyl group,
a C₅₋₆-cycloalkyl group wherein a methylene group is replaced by an oxygen or sulphur atom, by an imino group substituted by the group R⁴, by a sulphinyl or sulphonyl group, while R⁴ is as hereinbefore defined,
a hydroxy or C₁₋₄-alkoxy group, or also
a hydrogen atom, if n is the number 0, and
R^{c} denotes a hydrogen atom, a C₁₋₄-alkoxy-C₁₋₄-alkoxy, C₁₋₄-alkoxy, C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group,
a 3-pyrrolidinyloxy, 2-pyrrolidinyl-C₁₋₃-alkyloxy, 3-pyrrolidinyl-C₁₋₃-alkyloxy, 3-piperidinyloxy, 4-piperidinyloxy, 2-piperidinyl-C₁₋₃-alkyloxy, 3-piperidinyl-C₁₋₃-alkyloxy or 4-piperidinyl-C₁₋₃-alkyloxy group, wherein in each case the cyclic nitrogen atom is substituted by the group R⁴, where R⁴ is as hereinbefore defined,
a piperazino or homopiperazino group substituted in the 4 position by an R⁶—C₁₋₄-alkyl, R⁶—CO or R⁶—C₁₋₄-alkylene-CO group, wherein
R⁶ denotes a 2-oxo-tetrahydrofuranyl, 2-oxo-tetrahydropyranyl, 2-oxo-1,4-dioxanyl or 2-oxo-4—(C₁₋₄-alkyl)-morpholinyl group optionally substituted by one or two C₁₋₂-alkyl groups,
a morpholino-C₁₋₄-alkoxy or 2-oxo-morpholin-4-yl-C₁₋₆-alkoxy group which may be substituted by 1 or 2 methyl or ethyl groups, or
a tetrahydrofuran-3-yloxy, tetrahydropyran-3-yloxy, tetrahydropyran-4-yloxy, tetrahydrofuranylmethoxy or tetrahydropyranylmethoxy group,
(b) the compounds
(1) 4-[(3-chloro-4-fluorophenyl)amino]-6-[(4-dimethylamino-cyclohexyl)amino]-pyrimido[5,4-d]pyrimidine,
(2) 4-[(R)-(1-phenylethyl)amino]-6-(4-hydroxyphenyl)-7H-pyrrolo[2,3-d]pyrimidine,
(3) 4-{[3-chloro-4-(3-fluoro-4-benzyloxy)-phenyl]amino}-6-(5-{[(2-methanesulphonyl-ethyl)amino]methyl}-furan-2-yl)quinazoline or
as well as tautomers and pharmaceutically acceptable salts of one of the foregoing substances,
(c) the antibodies
(1) Cetuximab C225, Trastuzumab,
(2) ABX-EGF, and
(3) Mab ICR-62, and
(d) EGFR-antisense.

3. The method according to claim 1, wherein the substance administered is selected from the group consisting of:
(a) compounds of the formula I, wherein:
X denotes a nitrogen atom or a carbon atom substituted by a cyano group,
R^{a} denotes a hydrogen atom,
R^{b} denotes a phenyl or 1-phenylethyl group, wherein the phenyl nucleus in each case is substituted by the groups R¹ and R², while
R¹ and R², which may be identical or different, in each case denote a hydrogen, fluorine, chlorine or bromine atom,
a C₁₋₄-alkyl, C₂₋₅-alkenyl or C₂₋₅-alkynyl group,
A denotes an oxygen atom or an imino group,
B denotes a bond or a carbonyl group,
C denotes a methylene, ethylene or ethenylene group,
n denotes one of the numbers 0 or 1,
D denotes a di-(C₁₋₄-alkyl)-amino group wherein the alkyl moieties may be identical or different,
a methylamino or ethylamino group, wherein in each case the nitrogen atom is substituted by a 2-methoxyethyl, tetrahydrofuran-3-yl, tetrahydropyran-4-yl, tetrahydrofuran-2-ylmethyl, cyclopropyl or cyclopropylmethyl group,
an N—(C₁₋₄-alkyl)—N—(C₂₋₄-alkyl)-amino group wherein the alkyl moieties in the β, γ or δ position to the nitrogen atom of the amino group may optionally be substituted by the group R³, while
R³ denotes a C₁₋₃-alkoxy or C₁₋₃-alkoxycarbonyl group,
a bis-(2-methoxyethyl)-amino group,
a morpholino or 2-oxo-morpholin-4-yl group optionally substituted by a methyl or methoxymethyl group,
a hydroxy or C₁₋₄-alkoxy group, or also
a hydrogen atom, if n is the number 0, and
R^{c} denotes a hydrogen atom, a C₄₁₋-alkoxy-C₁₋₄-alkoxy, C₁₋₄-alkoxy, C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group,
a 3-pyrrolidinyloxy, 2-pyrrolidinyl-C₁₋₃-alkyloxy, 3-pyrrolidinyl-C₁₋₃-alkyloxy, 3-piperidinyloxy, 4-piperidinyloxy, 2-piperidinyl-C₁₋₃-alkyloxy, 3-piperidinyl-C₁₋₃-alkyloxy or 4-piperidinyl-C₁₋₃-alkyloxy group, wherein in each case the cyclic nitrogen atom is substituted by the group R⁴, where R⁴ is as hereinbefore defined,
a piperazino or homopiperazino group substituted in the 4 position by an R⁶—C₁₋₄-alkyl, R⁶—CO or R⁶—C₁₋₄-alkylene-CO group, wherein
R⁶ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups,
a morpholino-C₁₋₄-alkoxy or 2-oxo-morpholin-4-yl-C₁₋₆-alkoxy group which may be substituted by 1 or 2 methyl or ethyl groups, or
a tetrahydrofuran-3-yloxy, tetrahydropyran-3-yloxy, tetrahydropyran-4-yloxy, tetrahydrofuranylmethoxy or tetrahydropyranylmethoxy group,
(b) the compounds
(1) 4-[(3-chloro-4-fluorophenyl)amino]-6-[(4-dimethylamino-cyclohexyl)amino]-pyrimido[5,4-d]pyrimidine,
(2) 4-[(R)-(1-phenylethyl)amino]-6-(4-hydroxyphenyl)-7H-pyrrolo[2,3-d]pyrimidine, and
(3) 4-{[3-chloro-4-(3-fluoro-4-benzyloxy)-phenyl]amino}-6-(5-{[(2-methanesulphonyl-ethyl)amino]methyl}-furan-2-yl)quinazoline,
as well as tautomers and pharmaceutically acceptable salts of one of the foregoing substances,
(c) the antibodies
(1) Cetuximab C225,
(2) Trastuzumab, ABX-EGF, and
(3) Mab ICR-62
(d) EGFR-antisense.

4. The method according to claim 1, wherein the substance administered is selected from the group consisting of:
(1) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,
(2) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,
(3) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,
(4) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,
(5) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline
(6) 4-[(3-chloro-4-fluorophenyl)amino]-6-{[4-(morpholin-4-yl)-1-oxo-2-buten-1-yl]-amino}-7-cyclopropylmethoxy-quinazoline,
(7) 4-[(3-chloro-4-fluorophenyl)amino]-6-{[4-(N,N-diethylamino)-1-oxo-2-buten-1-yl]amino}-7-cyclopropylmethoxy-quinazoline,
(8) 4-[(3-chloro-4-fluorophenyl)amino]-6-{[4-(N,N-dimethylamino)-1-oxo-2-buten-1-yl]amino}-7-cyclopropylmethoxy-quinazoline,
(9) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,
(10) 4-[(R)-(1-phenyl-ethyl)amino]-6-{[4-(morpholin-4-yl)-1-oxo-2-buten-1-yl]amino}-7-cyclopropylmethoxy-quinazoline,
(11) 4-[(R)-(1-phenyl-ethyl)amino] -6-{[4-(morpholin-4-yl)-1-oxo-2-buten-1-yl]amino}-7-cyclopentyloxy-quinazoline,
(12) 4-[(3-chloro-4-fluoro-phenyl)amino]-6-{[4-((R)-6-methyl-2-oxo-morpholin-1-oxo-2-buten-1-yl]amino}-7-cyclopropylmethoxy-quinazoline,
(13) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline
(14) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline,
(15) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,
(16) 4-[(3-chloro-4-fluoro-phenyl)amino]-6-[2-((S)-6-methyl-2-oxo-morpholin-4-yl)-ethoxy]-7-methoxy-quinazoline,
(17) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(18) 4-[(3-chloro-4-fluorophenyl)amino]-6-{[4-(N,N-dimethylamino)-1-oxo-2-buten-1-yl]amino}-7-cyclopentyloxy-quinazoline,
(19) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,
(20) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,
(21) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,
(22) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,
(23) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,
(24) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(25) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(26) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(27) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(28) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(29) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(30) 4-[(3-chloro-4-fluorophenyl)amino]-6-[3-(morpholin-4-yl)-propyloxy]-7-methoxy-quinazoline,
(31) 4-[(3-ethynyl-phenyl)amino]-6,7-bis-(2-methoxy-ethoxy)-quinazoline,
(32) 4-[(3-chloro-4-fluorophenyl)amino]-7-[3-(morpholin-4-yl)-propyloxy]-6-[(vinyl-carbonyl)amino]-quinazoline,
(33) 4-{[3-chloro-4-(3-fluoro-benzyloxy)-phenyl]amino}-6-(5-{[(2-methanesulphonyl-ethyl)amino]methyl}-furan-2-yl)quinazoline,
(34) 4-[(3-chloro-4-fluoro-phenyl)amino]-6-[(4-dimethylamino-cyclohexyl)amino]-pyrimido[5,4-d]pyrimidine,
(35) 4-[(R)-(1-phenyl-ethyl)amino]-6-(4-hydroxy-phenyl)-7H-pyrrolo[2,3-d]pyrimidine,
(36) 3-cyano-4-[(3-chloro-4-fluorophenyl)amino]-6-{[4-(N,N-dimethylamino)-1-oxo-2-buten-1-yl]amino}-7-ethoxy-quinoline,
as well as the pharmaceutically acceptable salts of the foregoing,
(37) Cetuximab,
(38) Trastuzumab,
(39) antibody ABX-EGF,
(40) Mab ICR-62, and
(41) EGFR-antisense.

5. The method according to claim 1, wherein the substance administered is selected from the group consisting of:
(1) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,
(2) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,
(3) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,
(4) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,
(5) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline,
(6) 4-[(3-chloro-4-fluorophenyl)amino]-6-{[4-(morpholin-4-yl)-1-oxo-2-buten-1-yl]-amino}-7-cyclopropylmethoxy-quinazoline,
(7) 4-[(3-chloro-4-fluorophenyl)amino]-6-{[4-(N,N-diethylamino)-1-oxo-2-buten-1-yl]amino}-7-cyclopropylmethoxy-quinazoline,
(8) 4-[(3-chloro-4-fluorophenyl)amino]-6-{[4-(N,N-dimethylamino)-1-oxo-2-buten-1-yl]amino}-7-cyclopropylmethoxy-quinazoline,
(9) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,
(10) 4-[(R)-(1-phenyl-ethyl)amino]-6-{[4-(morpholin-4-yl)-1-oxo-2-buten-1-yl]amino}-7-cyclopropylmethoxy-quinazoline,
(11) 4-[(R)-(1-phenyl-ethyl)amino]-6-{[4-(morpholin-4-yl)-1-oxo-2-buten-1-yl]amino}-7-cyclopentyloxy-quinazoline,
(12) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,
(13) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline,
(14) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline,
(15) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,
(16) 4-[(3-chloro-4-fluoro-phenyl)amino]-6-[2-((S)-6-methyl-2-oxo-morpholin-4-yl)-ethoxy]-7-methoxy-quinazoline,
(17) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(18) 4-[(3-chloro-4-fluorophenyl)amino]-6-{[4-(N,N-dimethylamino)-1-oxo-2-buten-1-yl]amino}-7-cyclopentyloxy-quinazoline,
(19) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,
(20) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,
(21) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,
(22) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,
(23) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,
(24) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(25) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(26) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(27) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(28) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(29) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(30) 4-[(3-chloro-4-fluorophenyl)amino]-6-[3-(morpholin-4-yl)-propyloxy]-7-methoxy-quinazoline and the compound, and
(30) 4-[(3-chloro-4-fluoro-phenyl)amino]-6-[(4-dimethylamino-cyclohexyl)amino]-pyrimido[4-d]pyrimidine
and the pharmaceutically acceptable salts thereof.

6. The method according to claim 1, wherein the substance administered is selected from the group consisting of:
(1) 4-[(3-chloro-4-fluoro-phenyl)amino]-7-[4-((R)-6-methyl-2-oxo-morpholin-4yl)-butyloxy]-6-[(vinylcarbonyl)amino]-quinazoline,
(2) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,
(3) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,
(4) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,
(5) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-metboxy-quinazoline,
(6) 4-[(R)-(1-phenyl-ethyl)amino]-6-{[4-(morpholin-4-yl)-1-oxo-2-buten-1-yl]}-7-cyclopropylmethoxy-quinazoline, and
(7) 4-[(3-chloro-4-fluorophenyl)amino]-6-[3-(morpholin-4-yl)-propyloxy]-7-methoxy-quinazoline
and the pharmaceutically acceptable salts thereof.

7. The method of claim 1, 3, 4, 5 or 6 wherein the condition to be treated is COPD, chronic sinusitis, asthma, cystic fibrosis, Crohn's disease, ulcerative colitis or polyposis of the intestines.

8. The method of claim 1, 3, 4, 5 or 6 wherein the condition to be treated is COPD, asthma or cystic fibrosis.
